SEQUENCE LISTING (2 sequences)

SEQ ID NO: 1 - DNA, 1398 bp, Heliothis virescens
  CDS (1)..(1398)
atgtccgtggcgaagaaagacaagccgacaatgtcggtgacagcacttatcaactgggctcgacccttgccgccgggccaacagcagcagccgatgacgcctacgtcgcccggaaacatgcttcaaccgatggctacgccgtctaacttaccgactgtcgactgctcactcgatattcaatggctaaacttggagggaggttttatgtcgccgatgtcaccgccggagatgaagccagacacggcgatgctagacggcctgcgagacgactccaccccacccccagctttcaagaactaccccccgaaccatcccctaagtggttctaagcacctctgttctatatgtggagatagagcgtcggggaaacattatggagtatacagttgtgaaggttgcaaaggtttcttcaaaaggacggtaagaaaagacttaacgtacgcatgccgcgaagaacgtaactgcatcatagacaaacgccagaggaacagatgccagtactgtaggtaccagaaatgtctcgcgtgcggcatgaagagggaagcggtgcaggaggagaggcagagggccgccagaggtacggaggatgcacatccgagcagctcggtgcaggtacaggagttatcaatcgagcggttgctggagatggagtcactggtagctgaccccagcgaagagttccagttccttcgtgtgggacccgacagtaatgtgccgcctaagttccgcgcccctgtctccagcctttgtcaaataggcaacaaacaaatagcggcgctagtggtgtgggcgcgcgacatcccgcacttcagccagcttgagatggaagaccagatcctgctcatcaaaggctcctggaacgaactgctgctcttcgccattgcgtggcggtctatggagttcctgacagaagagcgagacggcgtggacggcactgggaacagaaccacatcgccgccacaacttatgtgtctcatgcctggcatgacgctgcaccgcaactcagcgctgcaggcgggcgtggggcagatcttcgaccgcgtgctgtcggagctgtcgctgaagatgcgcaccctgcgcgtcgaccaggccgagtacgtcgcgctcaaggccatcatactgctcaacccagatgtgaagggactgaaaaacaggcaagaagtggaagttttacgagaaaagatgttcctgtgcctggacgagtactgccgccgctcgcgcagttcggaggagggtcggttcgcggcgctgctgctgcgcctgcccgcgttacgttccatttcactcaagagcttcgagcacctgttcttcttccacctggtggccgacaccagcatcgccggctacatccgcgacgcgctgcgcaaccacgcgccgcccatcgacaccaacatgatg

SEQ ID NO: 2 - protein, 466 aa, Heliothis virescens
MSVAKKDKPTMSVTALINWARPLPPGQQQQPMTPTSPGNMLQPMATPSNLPTVDCSLDIQWLNLEGGFMSPMSPPEMKPDTAMLDGLRDDSTPPPAFKNYPPNHPLSGSKHLCSICGDRASGKHYGVYSCEGCKGFFKRTVRKDLTYACREERNCIIDKRQRNRCQYCRYQKCLACGMKREAVQEERQRAARGTEDAHPSSSVQVQELSIERLLEMESLVADPSEEFQFLRVGPDSNVPPKFRAPVSSLCQIGNKQIAALVVWARDIPHFSQLEMEDQILLIKGSWNELLLFAIAWRSMEFLTEERDGVDGTGNRTTSPPQLMCLMPGMTLHRNSALQAGVGQIFDRVLSELSLKMRTLRVDQAEYVALKAIILLNPDVKGLKNRQEVEVLREKMFLCLDEYCRRSRSSEEGRFAALLLRLPALRSISLKSFEHLFFFHLVADTSIAGYIRDALRNHAPPIDTNMM

(19) United States
(12) Patent Application Publication
(10) Pub. No.: US 2002/0037556 A1
(43) Pub. Date: Mar. 28, 2002

(54) Heliothis virescens ultraspiracle (USP) protein

(76) Inventors: Werner Zitzmann, Leverkusen (DE); Eva-Maria Franken, Leichlingen (DE); Martina Janssen, Konigswinter (DE); Thomas Schulte, Koln (DE)

Correspondence Address: BAYER CORPORATION, PATENT DEPARTMENT, 100 BAYER ROAD, PITTSBURGH, PA, 15205, US

(21) Appl. No.: 09/909,672

(22) Filed: Jul. 20, 2001

(30) Foreign Application Priority Data
Jul. 25, 2000 (DE) 10036469.1

(51) Int. Cl. C12P 21/02; C12Q 1/00; C07H 21/04; C12N 9/00; C12N 5/06; C12N 1/21; C12N 5/04

(52) U.S. Cl. 435/69.1; 435/4; 435/183; 435/252.33; 435/348; 435/410; 536/23.2

(57) ABSTRACT

The invention relates to nucleic acids which encode polypeptides with the bioactivity of the ultraspiracle protein, and to such polypeptides per se. The invention furthermore relates to methods of finding insecticidal active compounds and for the controlled expression of target genes (gene switch).

The invention relates to nucleic acids which encode polypeptides with the bioactivity of the ultraspiracle protein, and to such polypeptides per se. The invention furthermore relates to methods of finding insecticidal active compounds and for the controlled expression of target genes (gene switch).

The ultraspiracle protein (termed USP hereinbelow) is the insect ortholog of the vertebrate retinoid X receptor (RXR). Like RXR, it belongs to the family of the nuclear receptors. These nuclear receptors are located inside the cell. They bind to responsive elements on the DNA as homodimers or heterodimers and regulate the expression of genes. In order to be active, they must bind specific small hydrophobic ligands (for example steroids, retinoids, vitamin D). Nuclear receptors have a modular structure with functional domains for transactivation, DNA binding and ligand binding. The DNA binding domain contains a number of cysteine residues and forms a characteristic structure, termed the zinc finger.

Owing to their structural and functional properties (DNA binding to specific elements, activation of downstream genes), nuclear receptors are suitable as components for expression systems which can be regulated (gene switch). Some nuclear receptors (for example RXR, EcR) are already being used in inducible eukaryotic expression systems (Invitrogen Corporation, Carlsbad Calif., USA).

In insects, for example, the development from the larva to the adult insect is controlled via nuclear receptors, with the steroid hormone ecdysone and the isoprenoid juvenile hormone being involved (1;2;3;4). The ecdysone receptor, a nuclear receptor composed of two different subunits, EcR and USP, plays a key role (5;6;7). While the hormone ecdysone (in its active form 20-hydroxyecdysone) has been known for a long time as ligand for the EcR subunit, USP is an orphan receptor for which no ligand has been identifiable as yet.

The ecdysone receptor constitutes an important insecticide target. Its activation outside the time window provided for this purpose during insect development leads to severe disruptions or even to the death of the insect. This mechanism forms the basis for insecticidal ecdysone agonists (8;9). These are nonsteroidal ligands of the EcR subunit which act specifically on lepidopterans (10). Since the ecdysone/juvenile-hormone-controlled development is only found in invertebrates and does not occur in vertebrates, it constitutes an insecticidal mechanism which is safe for the user.

The protein sequence of a number of insect USPs is already known. Thus, for example, the sequences of Drosophila melanogaster, Manduca sexta, Choristoneura fumiferana and Bombyx mori have been described (11).

Since USP is an orphan receptor for which no ligand is known as yet, this receptor is of great practical importance for establishing screening systems for the search for new ligands which can then be used, inter alia, as insecticides. If ligands for USP are available, this nuclear receptor can be used in systems for the controlled expression of target genes (gene switch).

The present invention relates to nucleic acids which encode polypeptides with the bioactivity of USP and which comprise a sequence selected from:

a) the sequence of SEQ ID NO: 1,

b) sequences which have at least 85% identity, preferably at least 90% identity, especially preferably at least 95% identity, with the sequence of SEQ ID NO: 1 over a length of at least 600 consecutive nucleotides and preferably over their entire length,

c) sequences which, owing to the degeneracy of the genetic code, encode the same amino acid sequence as the sequences defined under (a) and (b),

d) parts of the sequences as defined under (a), (b) and (c) which encode polypeptides which have essentially the same bioactivity as a polypeptide with the amino acid sequence of SEQ ID NO: 2.

The degree of identity of the nucleic acid sequences is preferably determined using the program GAP from the program package GCG, Version 9.1, using standard settings.

The invention furthermore relates to vectors which contain at least one of the nucleic acids according to the invention. Vectors which can be used are all the plasmids, phasmids, cosmids, YACs or artificial chromosomes used in molecular biology laboratories. To express the nucleic acids according to the invention, they may be linked to customary regulatory sequences. The choice of such regulatory sequences depends on whether pro- or eukaryotic cells or cell-free systems are used for expression. Especially preferred as expression control sequence are, for example the SV40 or adenovirus or cytomegalovirus early or late promoters, the AcMNPV immediate early promoter, the lac system, the trp system, the main operator and promoter regions of phage lambda, the control regions of the fd coat protein, the 3-phosphoglycerate kinase promoter, the acid phosphatase promoter, the yeast α-mating factor promoter and the cauliflower mosaic virus 35S promoter. The term “promoter” as used in the present context relates generally to expression control sequences.

To express the nucleic acids according to the invention, they can be introduced into suitable host cells. The term “host cell” as used in the present context relates to cells which do not naturally contain the nucleic acids according to the invention. Suitable host cells are prokaryotic cells, preferably E. coli, and eukaryotic cells such as mammalian, insect and plant cells. Examples of suitable single-celled host cells are: Pseudomonas, Bacillus, Streptomyces, yeasts, HEK-293, Schneider S2, Sf9, CHO, COS 1, COS7 cells. However, cells which are components of complex systems (for example entire plants or animals) are also suitable. The present invention therefore also relates to transgenic organisms (with the exception of humans) such as, for example, plants and animals which contain the nucleic acids according to the invention. The term “transgenic” as used in the present context means that the nucleic acid according to the invention has been introduced into the organism by recombinant methods.

The present invention also relates to the polypeptides which are encoded by the nucleic acids according to the invention and to the receptors composed of them and consisting of an EcR subunit and a polypeptide according to the invention

The term “polypeptides” as used in the present context refers to short amino acid chains, which are usually termed peptides, oligopeptides or oligomers, and to long amino acid chains, usually termed proteins. It comprises amino acid chains which can be modified either by natural processes, such as post-translational processing, or by chemical prior art methods. Such modifications may occur at various sites and repeatedly in a polypeptide, such as, for example, at the peptide backbone, at the amino acid side chain, at the amino terminus and/or at the carboxy terminus. They comprise, for example, acetylations, acylations, ADP ribosylations, amidations, covalent linkages to flavins, haem moieties, nucleotides or nucleotide derivatives, lipids or lipid derivatives or phosphatidylinositol, cyclizations, the formation of disulphide bridges, demethylations, the formation of cystine, formylations, gamma-carboxylations, glycosylations, hydroxylations, iodinations, methylations, myristoylations, oxidations, proteolytic processings, phosphorylations, selenoylations and tRNA-mediated additions of amino acids.

The polypeptides according to the invention may exist in the form of “mature” proteins or as parts of larger proteins, for example as fusion proteins. They may furthermore have secretion or “leader” sequences, pro-sequences, sequences which allow simple purification such as multiple histidine residues, or additional stabilizing amino acids.

The bioactivity of the polypeptides according to the invention can be detected for example by a transactivation assay. To this end, a test polypeptide in combination with an EcR subunit and a reporter construct composed of a promoter with EcR binding sequence and a reporter gene is expressed in a cell system. If, in the presence of ecdysone or an ecdysone analogue, the reporter gene product can be detected, for example by an enzyme assay, this means that the polypeptide tested has the bioactivity of a polypeptide according to the invention.

Suitable reporter genes and binding sequences are described, for example, in WO 97/45737.

The polypeptides according to the invention need not constitute complete USPs, but may also just be fragments thereof as long as they still have at least the bioactivity of a polypeptide (USP) with the amino acid sequence of SEQ ID NO: 2. It is not necessary that the polypeptides according to the invention can be derived directly from a Heliothis virescens USP.

Compared with the corresponding region of a naturally occurring Heliothis virescens USP, the polypeptides according to the invention may exhibit deletions or amino acid substitutions as long as they still exert at least the bioactivity of a USP. Conservative substitutions are preferred. Such conservative substitutions encompass variations in which one amino acid is replaced by another amino acid from the following group:

1. Small aliphatic residues, unpolar residues or residues of little polarity: Ala, Ser, Thr, Pro and Gly;

2. Polar, negatively charged residues and their amides: Asp, Asn, Glu and Gln;

3. Polar, positively charged residues: His, Arg and Lys;

4. Large aliphatic unpolar residues: Met, Leu, Ile, Val and Cys; and

5. Aromatic residues: Phe, Tyr and Trp.

Preferred conservative substitutions can be seen from the following list:

| Original residue | Substitution |
|---|---|
| Ala | Gly, Ser |
| Arg | Lys |
| Asn | Gln, His |
| Asp | Glu |
| Cys | Ser |
| Gln | Asn |
| Glu | Asp |
| Gly | Ala, Pro |
| His | Asn, Gln |
| Ile | Leu, Val |
| Leu | Ile, Val |
| Lys | Arg, Gln, Glu |
| Met | Leu, Tyr, Ile |
| Phe | Met, Leu, Tyr |
| Ser | Thr |
| Thr | Ser |
| Trp | Tyr |
| Tyr | Trp, Phe |
| Val | Ile, Leu |

A preferred embodiment of the polypeptides according to the invention is a Heliothis virescens USP which has the amino acid sequence of SEQ ID NO: 2.

The invention furthermore relates to antibodies which bind specifically to the abovementioned polypeptides or receptors. Such antibodies are produced in the customary fashion. For example, such antibodies can be raised by injecting a substantially immunocompetent host with an amount of a polypeptide according to the invention or fragment thereof which is effective for antibody production, and subsequently obtaining this antibody. Furthermore, an immortalized cell line which produces monoclonal antibodies may be obtained in a manner known per se. If appropriate, the antibodies may be labelled with a detection reagent. Preferred examples of such a detection reagent are enzymes, radiolabelled elements, fluorescent chemicals or biotin. Instead of the complete antibody, fragments may also be employed which have the desired specific binding properties. The term “antibody” as used in the present context therefore also extends to parts of complete antibodies, such as Fa, F(ab′)₂ or Fv fragments, which are still capable of binding to the epitopes of the polypeptides according to the invention.

In order to produce the polypeptides which are encoded by the nucleic acids according to the invention, host cells which contain at least one of the nucleic acids according to the invention can be cultured under suitable conditions. Then, the desired polypeptides can be isolated from the cells or the culture medium in the customary manner.

A rapid method of isolating the polypeptides according to the invention which are synthesized by host cells using a nucleic acid according to the invention starts with expressing a fusion protein, it being possible for the fusion partner to be affinity-purified in a simple manner. The fusion partner may be, for example, glutathione S-transferase. The fusion protein can then be purified on a glutathione affinity column. The fusion partner can be removed by partial proteolytic cleavage, for example at linkers between the fusion partner and the polypeptide according to the invention to be purified. The linker can be designed such that it includes target amino acids such as arginine and lysine residues which define sites for trypsin cleavage. Standard cloning methods using oligonucleotides may be employed to generate such linkers.

Other purification methods which are possible are based on preparative electrophoresis, FPLC, BPLC (for example using gel filtration columns, reversed-phase columns or moderately hydrophobic columns), gel filtration, differential precipitation, ion-exchange chromatography or affinity chromatography.

The nucleic acids according to the invention can be prepared in the customary manner. For example, the nucleic acid molecules can be chemically synthesized in their entirety. Alternatively, short portions of the sequences according to the invention can be synthesized chemically, and such oligonucleotides can be radiolabelled or labelled with a fluorescent dye. The labelled oligonucleotides can be used for searching cDNA libraries generated on the basis of insect mRNA. Clones with which the labelled oligonucleotides hybridize are selected for isolating the DNA in question. After the isolated DNA has been characterized, the nucleic acids according to the invention are obtained in a simple fashion.

Additionally, the nucleic acids according to the invention can be prepared by PCR methods using chemically synthesized oligonucleotides.

The nucleic acids according to the invention can be used for isolating and characterizing the regulatory regions which naturally occur in the vicinity of the coding region. Thus, the present invention also relates to such regulatory regions.

The nucleic acids according to the invention allow the identification, by in vivo methods, of new ligands of the USP subunit of an ecdysone receptor. For example, a recombinant DNA molecule which comprises at least one nucleic acid according to the invention may be introduced into a suitable host cell for this purpose. The host cell is cultured in the presence of a chemical or a mixture of chemicals under conditions which allow the expression of the polypeptides according to the invention. Activation or inhibition of the receptor can be made detectable by transactivating a reporter gene (for example luciferase, beta-galactosidase) which is arranged downstream of a suitable promoter with USP binding sequence (12).

The nucleic acids according to the invention also allow compounds which bind to the polypeptides according to the invention to be found by means of in vitro methods. The polypeptides according to the invention can be contacted with a chemical or a mixture of chemicals under conditions which permit the interaction of at least one compound with the polypeptide according to the invention. The binding of compounds to a polypeptide according to the invention can be detected, for example, by the displacement of a radiolabelled or fluorescence-labelled ligand. A polypeptide according to the invention may also be labelled for this purpose, for example to allow a fluorescence resonance energy transfer (FRET) method to be applied.

Ligands found in this manner can be used in crop protection as new insecticidal substances. Such ligands can take the form of small organochemical molecules, peptides or antibodies.

A further application of the nucleic acids, vectors and regulatory regions according to the invention described hereinabove is their use as chemically inducible expression systems (gene switch) for a variety of target genes. To this end, the nucleic acids can be expressed in host cells as described above. The target genes are cloned into expression vectors which are provided with a suitable promoter with regulatory regions. These expression vectors are then also introduced into the host cells. The transcription of the target gene can be regulated by adding, to the host cells, a ligand as described above. An advantageous use, in addition to the use in cultured cells, is, in particular, the use in plants, since plants have no endogenous nuclear receptors and since no other well-functioning chemically inducible expression system is currently available for plants. The production of proteins in plants is very promising. However, therapeutic applications in animals, including humans, are also possible.

Information on the sequence listing:

SEQ ID NO: 1 shows the nucleotide sequence of the Heliothis virescens USP. SEQ ID NO: 2 shows the amino acid sequence of the protein derived from the Heliothis virescens USP nucleotide sequence.

### EXAMPLES

### Example 1

Isolation of the above-described polynucleotides

Polynucleotides were manipulated by standard methods of recombinant DNA technology (13). Nucleotide and amino acid sequences were processed in terms of bioinformatics using the program package GCG Version 9.1 (GCG Genetics Computer Group, Inc., Madison, Wis., USA).

The RNA for the cDNA library was isolated from entire Heliothis virescens larvae (2nd and 3rd instar) using Trizol reagent (Gibco BRL, following the manufacturer's instructions). From these RNAs, the poly-A-containing RNAs were then isolated by purification using Dyna Beads 280 (Dynal). 5 μg of these poly-A-containing RNAs were subsequently employed for constructing the cDNA library using the vector λ-ZAPExpress (cDNA Synthesis Kit, ZAP-cDNA Synthesis Kit and ZAP-cDNA Gigapack III Gold Cloning Kit, all from Stratagene). In a deviation from the manufacturer's instructions, Reverse Transcriptase Superscript (Gibco BRL) was used for synthesizing cDNA at a synthesis temperature of 45° C. Also, no radiolabelled deoxynucleoside triphosphates were added. Moreover, the cDNAs synthesized were not fractionated using the gel filtration medium which is part of the kit, but using Size Sep 400 Spun Columns (Pharmacia).

All screens were carried out with the aid of the DIG system (all reagents and consumables were from Boehringer Mannheim and the instructions in “The DIG System User's Guide for Filter Hybridization”, Boehringer Mannheim, were followed). The DNA probes employed were prepared by PCR using digoxygenin-labelled dUTP. The hybridizations were performed in DIG Easy Hyb (Boehringer Mannheim) at 40° C. overnight. Detection of labelled DNA on nylon membranes was by chemoluminescence (CDP-Star, Boehringer Mannheim) using X-ray films (Lumifilm, Boehringer Mannheim). For identification, the isolated plasmids from the gene library were subjected to incipient sequencing by means of T3 and T7 primers (ABI Prism Dye Terminator Cycle Sequencing Kit, ABI, using the ABI Prism 310 Genetic Analyzer). The complete polynucleotide sequences were determined by primer walking by means of cycle sequencing; contract sequencing was carried out by MediGene, Martinsried.

References:

1. Segraves W. A. (1994): Steroid Receptors and Other Transcription Factors in Ecdysone Response. Recent Progress in Hormone Research, 49, 167-195

2. Henrich V. C. & Brown N. E. (1995): Insect Nuclear Receptors: A Developmental and Comparative Perspective. Insect Biochem. Mol. Biol. 25 (8), 881-897

3. Thummel C. S. (1995): From Embryogenesis to Metamorphosis: The Regulation and Function of Drosophila Nuclear Receptor Superfamily Members. Cell 83, 871-877

4. Truman J. W. (1996): Ecdysis Control Sheds Another Layer. Science 271, 40-41

5. Yao T et al. (1993): Functional ecdysone receptor is the product of EcR and Ultraspiracle genes. Nature 366, 476-479

6. Hall B. L. & Thummel C. S. (1998): The RXR homolog Ultraspiracle is an essential component of the Drosophila ecdysone receptor. Development 125, 4709-4717

7. Lezzi M. et al. (1999): The Ecdysone Receptor Puzzle. Arch. Insect Biochem. Physiol. 41, 99-106

8. Mikitani K. (1996): Ecdysteroid Receptor Binding Activity and Ecdysteroid Agonist Activity at the Level of Gene Expression are Correlated with the Activity of Dibenzoyl Hydrazines in Larvae of Bombyx mori. J. Insect Physiol. 42 (10), 937-941

9. Dhadialla T. S. et al. (1998): New Insecticides with Ecdysteroidal and Juvenile Hormone Activity. Annu. Rev. Entomol. 43, 545-569

10. Sundaram M. et al. (1998): Basis for selective action of a synthetic molting hormone agonist, RH-5992 on lepidopteran insects. Insect Biochem. Mol. Biol. 28, 693-704

11. Oro A. E. et al. (1990): Relationship between the product of the Drosophila ultraspiracle locus and the vertebrate retinoid X receptor. Nature 347, 298-301

12. Vögtli M. et al. (1998): High level transactivation by the ecdysone receptor complex at the core recognition motif. Nucl. Acid Res. 26 (10), 2407-2414

13. Sambrook et al. (1989): Molecular Cloning, A Laboratory Manual, 2nd ed. Cold Spring Harbor Press

1. Nucleic acid encoding a polypeptide with the bioactivity of the ultraspiracle protein, comprising a sequence selected from
(a) the sequence of SEQ ID NO: 1,
(b) sequences which have at least 85% identity with the sequence of SEQ ID NO: 1 over a length of at least 600 consecutive nucleotides,
(c) sequences which, owing to the degeneracy of the genetic code, encode the same amino acid sequence as the sequences defined under (a) and (b),
(d) parts of the sequences as defined under (a), (b) and (c) which encode polypeptides which have essentially the same bioactivity as a polypeptide with the amino acid sequence of SEQ ID NO: 2.

2. Vector comprising at least one nucleic acid according to claim 1.

3. Vector according to claim 2, characterized in that the nucleic acid molecule is linked functionally to regulatory sequences which ensure the expression of the nucleic acid in pro- or eukaryotic cells.

4. Host cell containing a nucleic acid according to claim 1 or a vector according to claim 2 or 3.

5. Host cell according to claim 4, characterized in that it is a pro- or eukaryotic cell.

6. Host cell according to claim 5, characterized in that the prokaryotic cell is E. coli.

7. Host cell according to claim 5, characterized in that the eukaryotic cell is a yeast cell, mammalian cell, insect cell or plant cell.

8. Transgenic organism, with the exception of humans, containing a nucleic acid according to claim 1 or a vector according to claim 2 or 3.

9. Polypeptide which is encoded by a nucleic acid according to claim 1.

10. Receptor comprising an EcR subunit and a polypeptide according to claim 9.

11. Antibody which binds specifically to a polypeptide according to claim 9.

12. Process for the preparation of a polypeptide according to claim 9, comprising the following steps:
(a) culturing a host cell according to one of claims 4 to 7 under conditions which ensure the expression of the nucleic acid according to claim 1, and
(b) obtaining the polypeptide from the cells or the culture medium.

13. Process for the preparation of a nucleic acid according to claim 1, comprising the following steps:
(a) complete chemical synthesis in a manner known per se or
(b) chemically synthesizing oligonucleotides, labelling the oligonucleotides, hybridizing the oligonucleotides with DNA of an insect cDNA library, selecting positive clones and isolating the hybridizing DNA from positive clones, or
(c) chemical synthesis of oligonucleotides and amplification of the target DNA by means of PCR.

14. Regulatory region which naturally controls the transcription of a nucleic acid according to claim 1 in insect cells and which ensures specific expression.

15. Method of finding new active compounds for crop protection, in particular compounds which cause the activation or inhibition of a polypeptide according to claim 9 or a receptor according to claim 10, comprising the following steps:
(a) providing a host cell according to one of claims 4 to 7,
(b) culturing the host cell in the presence of a chemical or a mixture of chemicals, and
(c) detecting the activation or inhibition of the polypeptide or receptor.

16. Method of finding a compound which binds to a polypeptide according to claim 9, comprising the following steps:
(a) contacting a polypeptide according to claim 9 with a compound or a mixture of compounds under conditions which permit the interaction of the compound(s) with the polypeptide, and
(b) identifying the compound which binds specifically to the polypeptide.

17. Method for inducibly expressing target genes by means of a polypeptide according to claim 9, comprising the following steps:
(a) culturing a host cell according to one of claims 4 to 7 or providing a transgenic organism according to claim 8 under conditions which ensure the expression of the nucleic acid according to claim 1, where the host cell or the transgenic organism contains a target gene with suitable regulatory sequences, and
(b) contacting the host cell or the transgenic organism with a chemical which induces the expression of the target gene.

18. Use of at least one nucleic acid according to claim 1, of a vector according to claim 2 or 3, of a host cell according to one of claims 4 to 7, of a transgenic organism according to claim 8, of a polypeptide according to claim 9, of a receptor according to claim 10 or of a regulatory region according to claim 14 for finding new active compounds for crop protection.

19. Use of at least one nucleic acid according to claim 1, of a vector according to claim 2 or 3, of a host cell according to one of claims 4 to 7, of a transgenic organism according to claim 8, of a polypeptide according to claim 9, of a receptor according to claim 10, of a regulatory region according to claim 14 or of a method according to claim 17 for the directed modification of the biological properties of a host cell or a host organism.
